(19) United States
(12) Patent Application Publication
(10) Pub. No.: US 2006/0178562 A1
(43) Pub. Date: Aug. 10, 2006

(54) Apparatus and methods for obtaining endoluminal access with a steerable guide having a variable pivot

(75) Inventors: Vahid Saadat, Saratoga, CA (US); Chris Rothe, San Jose, CA (US)

Correspondence Address: LEVINE BAGADE HAN LLP, 2483 EAST BAYSHORE ROAD, SUITE 100, PALO ALTO, CA, 94303, US

(73) Assignee: USGI Medical Inc., San Clemente, CA (US)

(21) Appl. No.: 11/057,042

(22) Filed: Feb. 10, 2005

(51) Int. Cl. A61B 1/00

(52) U.S. Cl. 600/142; 600/144; 600/146

(57) ABSTRACT

Methods and apparatus for obtaining endoluminal access are provided comprising a steerable endoluminal guide having a variable pivot for altering steering dynamics. In one variation, variable pivoting is achieved by advancing a member having a hard or substantially rigid section to various depths within a lumen of the guide. The hard section resists deformation, thereby reducing or precluding pivoting of the guide proximal of the distal-most depth of hard section insertion. In another variation, the guide comprises one or more pivot wires that terminate at a segment of the guide disposed proximal of the guide's distal outlet. In such a variation, the steering pivot of the guide may be altered by applying tension to at least one pivot wire, while concurrently applying tension to a steering wire that terminates more distally than the pivot wire, and that is radially offset from the pivot wire about the circumference of the guide. In another variation, one or more stiffening elements are translated over or alongside one or more steering wires to alter the guide's pivot.

### BACKGROUND OF THE INVENTION

### Field of the Invention

The present invention relates to methods and apparatus for endoluminally accessing gastrointestinal (“GI”) tissue. More particularly, the present invention relates to a steerable endoluminal guide having a variable pivot.

Morbid obesity is a serious medical condition pervasive in the United States and other countries. Its complications include hypertension, diabetes, coronary artery disease, stroke, congestive heart failure, multiple orthopedic problems and pulmonary insufficiency with markedly decreased life expectancy.

Several surgical techniques have been developed to treat morbid obesity, e.g., bypassing an absorptive surface of the small intestine, or reducing the stomach size. These procedures are difficult to perform in morbidly obese patients because it is often difficult to gain access to the digestive organs. In particular, the layers of fat encountered in morbidly obese patients make difficult direct exposure of the digestive organs with a wound retractor, and standard laparoscopic trocars may be of inadequate length. Furthermore, previously known open surgical procedures may present numerous life-threatening post-operative complications, and may cause atypical diarrhea, electrolytic imbalance, unpredictable weight loss and reflux of nutritious chyme proximal to the site of the anastomosis.

Applicant has previously described methods and apparatus for performing endoluminal gastroplasty, for example, in U.S. patent applications Ser. Nos. 10/734,562, filed Dec. 12, 2003; 10/841,415, filed May 7, 2004; 10/841,233, filed May 7, 2004; 10/916,768, filed Aug. 11, 2004; and Ser. No. 10/955,245, filed Sep. 29, 2004, all of which are incorporated herein by reference in their entireties. These methods typically rely on a multi-segmented endoluminal guide that is steered and/or retroflexed to a position wherein a distal outlet of the guide faces a body section of the guide. However, maintaining a substantially constant or fixed angle between a distal region of the guide and the body of the guide is difficult to achieve when working in multiple planes by retroflexing the guide to varying degrees. It therefore would be desirable to provide a steerable endoluminal guide having a variable pivot.

### BRIEF SUMMARY OF THE INVENTION

These and other objects of the present invention are accomplished by providing a steerable endoluminal guide having a variable pivot for altering steering dynamics. In one variation, the steerable guide comprises a plurality of discrete, nested segments interconnected by a plurality of tensioning wires radially disposed about each segment. The guide may be rigidized or shape-locked in any desired configuration by compressing the segments together via a radially balanced force applied to the segments through the tensioning wires. Likewise, the guide may be steered by selectively applying radially unbalanced forces to the segments via one or more of the wires, i.e., by applying a bending moment to the guide along the longitudinal axis.

The guide preferably is steerable into a retroflexed configuration wherein a distal outlet of the guide faces back towards a body section of the guide. As described in Applicant's co-pending U.S. patent application Ser. No. 10/734,562, filed Dec. 12, 2003, which previously has been incorporated herein by reference; in order to create a gastric pouch, tissue folds may need to be formed, approximated and secured in multiple planes. It is expected that a degree of retroflexion of the guide may need to be varied in order to visualize and/or engage gastric tissue in multiple planes for formation of the pouch. However, instruments configured for use with the guide to form the pouch, e.g., to engage tissue and form, secure and/or approximate tissue folds, may require or desire a substantially constant or fixed angle between the distal outlet or a distal region of the guide and its body to function adequately or optimally.

The present invention facilitates maintenance of a substantially fixed angle between the outlet or distal region of the guide and the body of the guide by providing the guide with a dynamically variable pivot for altering the dynamics of retroflexion or steering. In one variation, the variable pivot is achieved by advancing a member having a hard or substantially rigid section to various depths within a lumen of the guide. The hard section resists deformation, thereby reducing or precluding pivoting of the guide proximal of the distal-most depth of hard section insertion.

In an alternative variation, the guide comprises one or more tensioning pivot wires that terminate at a segment of the guide disposed proximal of the distal outlet. In such a variation, the steering pivot of the guide may be altered by applying tension to one of the pivot wires, while concurrently applying tension to a steering wire that terminates more distally than the pivot wire, and that is radially offset from the pivot wire about the circumference of the guide. When the steering wire is 180° offset from the pivot wire, it is expected that the guide will pivot or retroflex about the termination of the pivot wire. Pivot wires may terminate at various positions along the length of the guide to facilitate variable pivoting about the various positions.

Additional variations will be apparent to those of skill in the art. Methods of using the apparatus of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a schematic view of a variation of a steerable guide of the present invention;

FIG. 2 is a schematic detail view of discrete segments and tensioning wires of the steerable guide of FIG. 1;

FIGS. 3A and 3B are schematic views of the guide of FIG. 1 disposed within a patient's stomach, illustrating exemplary configurations in which it may be desirable to steer the guide;

FIG. 4 is a schematic view of a member that may be utilized in combination with the guide of FIG. 1 to vary a steering pivot of the guide;

FIGS. 5A-5C are side views, partially in section, illustrating a method of varying the pivot of the guide of FIG. 1 via the member of FIG. 4;

FIG. 6 is a schematic view of a variation of the steerable guide of FIG. 1 comprising integrally formed elements for varying a steering pivot;

FIG. 7 is a schematic detail view of the guide of FIG. 6, illustrating discrete segments, tensionable steering/rigidizing wires, and tensionable variable pivot wire(s) of the guide; and

FIGS. 8A-8C are schematic side views, partially in section, illustrating a method of using the apparatus of FIGS. 6 and 7 to form a gastric partition.

FIG. 9 is a schematic view of another variation of the steerable guide of FIG. 1 comprising alternative integrally formed elements for varying a steering pivot.

FIG. 10 is a schematic detail view of the guide of FIG. 9, illustrating discrete segments, tensionable steering/rigidizing wires and stiffening elements coaxially disposed over the wires.

### DETAILED DESCRIPTION OF THE INVENTION

The present invention relates to methods and apparatus for intraluminally accessing gastrointestinal (“GI”) tissue. More particularly, the present invention relates to a steerable endoluminal guide having a variable pivot for altering steering dynamics.

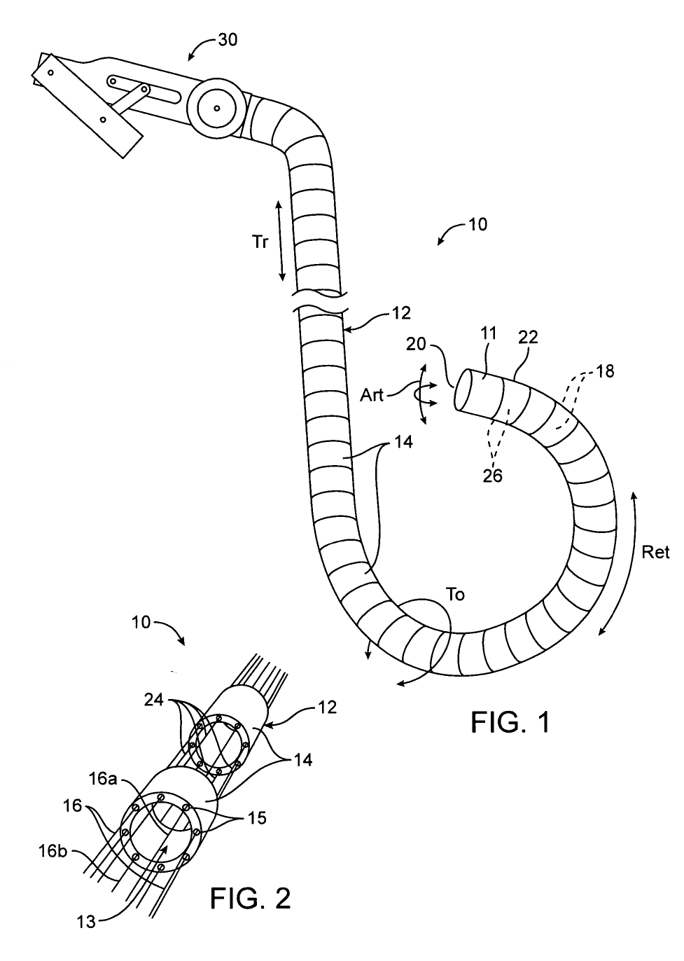

With reference to FIGS. 1 and 2, a variation of the guide is described. Apparatus 10 comprises steerable guide 12 having a plurality of discrete, nested segments 14 interconnected by a plurality of tensioning wires 16 radially disposed about each segment and passing through wire lumens 15 formed in each segment 14. Segments 14 further comprise central holes that when combined with adjacent segments 14 form one or more lumens 13, which extends through guide 12.

An interior liner (not shown) may extend through lumen(s) 13 and cover the interior wall of guide 12 to facilitate insertion and removal of instruments through the lumen of the guide, while a cover (not shown) may envelope the exterior wall of the guide to facilitate insertion and removal of the guide through a body lumen or cavity. The cover and/or liner optionally may comprise a hydrophilic coating, and optionally may be integrated into a single element within which the guide is disposed, embedded, encapsulated, etc.

Apparatus 10 also may comprise optional atraumatic tip 11 disposed at the distal end of guide 12. The atraumatic tip may reduce pinching or tearing of tissue when guide 12 is advanced within a body lumen or cavity, for example, over a diagnostic or therapeutic instrument. The tip may be fabricated from a variety of materials, for example, a tubular foam material. Atraumatic tip 11 may be coupled to the cover and liner; and the cover, liner and/or tip may be disposable.

Wires 16 extend proximally of segments 14, for example, to tensioning or steering control handle 30, and terminate distally at terminations 18. The distal terminations preferably comprise attachments to a segment of the guide disposed in the vicinity of distal outlet 20 of lumen(s) 13. The guide may be rigidized or shape-locked up to the point of terminations 18 in any desired configuration by compressing segments 14 together via a radially balanced force applied to the segments through tensioning wires 16.

Likewise, guide 12 may be steered by selectively applying radially unbalanced forces to segments 14 via one or more wires 16. For example, tension may be applied only to wire 16a, i.e., wire 16a may be retracted relative to guide 12, e.g., via handle 30, while the other wires 16 are not tensioned or retracted. This applies a bending moment to guide 12 along its longitudinal axis that achieves retroflexion Ret. A degree of retraction or advancement of wire 16a relative to guide 12 may be varied to alter a degree of retroflexion Ret of the guide. More complicated steering motions, such as four-way articulation Art of distal tip 22 of the guide, may, for example, be achieved by having one or more steering wires 24 that pass through wire lumens 15 and terminate within guide 12 at terminations 26 disposed distal of terminations 18, and/or by applying tension to wires 16 and 24 in various combinations.

As illustrated by arrows in FIG. 1, guide 12 also may be steered by a medical practitioner without tensioning of wires 16, for example, by advancing or retracting handle 30 to achieve translation Tr. Furthermore, the medical practitioner may rotate guide 12 while the guide is or is not shape-locked/rigidized in order to apply torque To the guide. The cover and/or liner optionally may comprise a torqueable skin for transmitting torque along guide 12. Additional steering techniques will be apparent to those of skill in the art.

As further illustrated in FIG. 1, guide 12 preferably is steerable into a retroflexed configuration wherein distal outlet 20 of the guide faces back towards a body section of the guide. As described in Applicant's co-pending U.S. patent application Ser. No. 10/734,562, filed Dec. 12, 2003, which previously has been incorporated herein by reference; in order to create a gastric pouch or partition, tissue folds may need to be formed, approximated and secured in multiple planes within a patient's stomach. It is expected that a degree of retroflexion Ret of guide 12 may need to be varied in order to visualize and/or engage gastric tissue in multiple planes for formation of the pouch or partition. However, instruments configured for use with the guide to form the pouch or partition, e.g., to engage tissue and form, secure and/or approximate tissue folds, may require or desire a substantially constant or fixed angle between distal outlet 20 or a distal region of guide 12 and the body of the guide in order to function adequately or optimally.

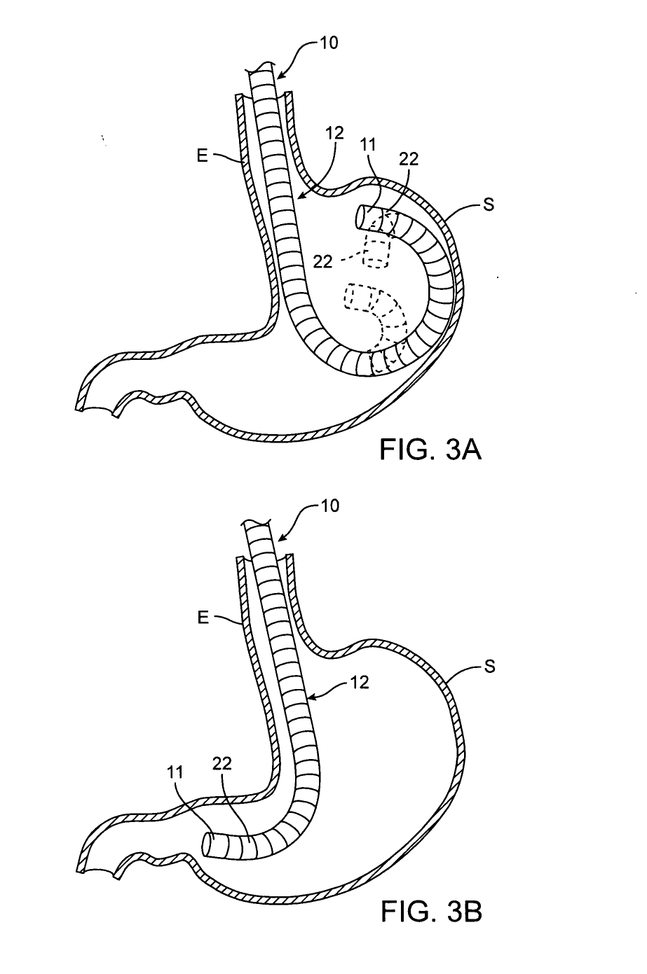

With reference to FIGS. 3, guide 12 of apparatus 10 is disposed within a patient's stomach to illustrate exemplary configurations in which it may be desirable to steer the guide. The guide may be advanced through esophagus E into stomach S in a substantially straight configuration, e.g., a straightened configuration or a configuration which conforms generally to the anatomy of the esophagus E. Guide 12 then may be steered or retroflexed to the configuration of FIG. 3A. As illustrated in dotted profile, it may be desirable to articulate tip 22 of the guide. Furthermore, as also shown in dotted profile, it may be desirable to vary the pivot of guide 12 to alter the dynamics of retroflexion, e.g., to access the gastric lumen at various locations with the distal region of guide 12 directed toward the body of guide 12 at a substantially fixed angle; exemplary methods and apparatus for dynamically altering the pivot of a steerable guide are described hereinafter. As seen in FIG. 3B, it also may be desirable to vary a direction of retroflexion or steering, for example, by retracting a wire 16b of FIGS. 1 and 2 disposed radially opposite wire 16a. Additional steering configurations will be apparent.

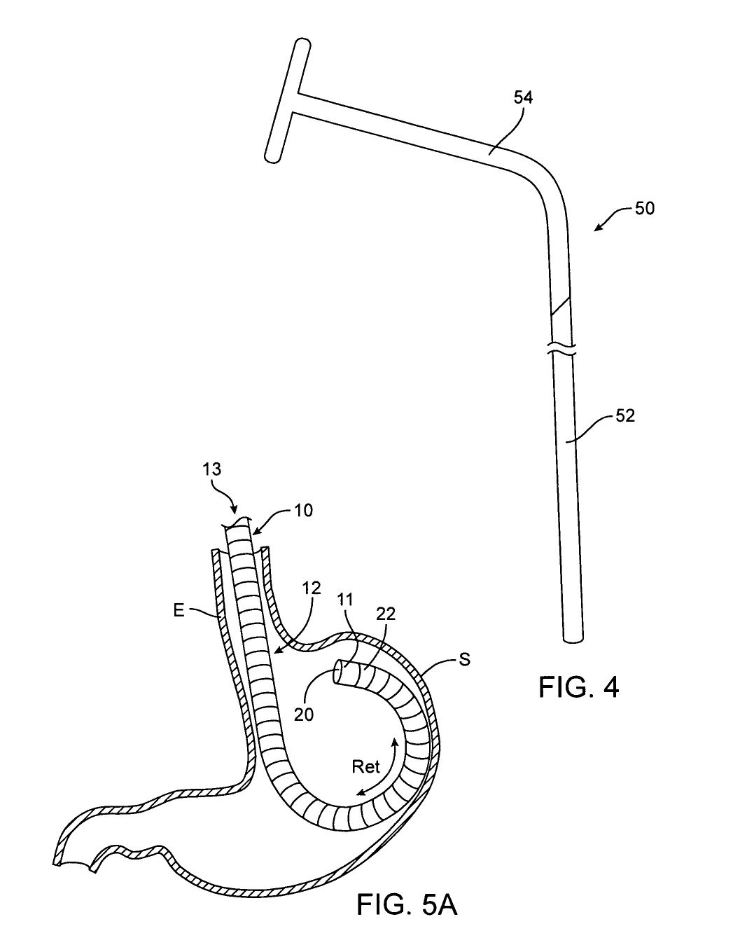

Referring now to FIG. 4, member 50 may be utilized in combination with apparatus 10 to dynamically vary a pivot of guide 12 for altering the dynamics of retroflexion or steering of the pivot. Member 50 comprises hard or substantially rigid distal segment 52, i.e., more hard, stiff, or rigid relative to a material of proximal segment 54, as well as proximal segment 54, which preferably is configured to conform to a patient's anatomy. Member 50 is sized for passage through lumen 13 and is fabricated from any number of suitable materials. For instance, member 50 may be fabricated from a singular material, e.g., stainless steel, Nitinol, plastics, etc., having a uniform hardness or stiffness along its entire length. Member 50 may also be fabricated from a singular material but have varying stiffness along its length, e.g., distal segment 52 having a greater stiffness relative to proximal segment 54. Variability in stiffness may be accomplished by a number of methods, e.g., in the case of member 50 being fabricated from a metallic material, the appropriate portions of member 50 may be alloyed to effect a suitable stiffness. Alternatively, geometry of member 50 may be varied along its length to affect stiffness. For example, distal segment 52 may have a first diameter while proximal segment 54 may have a second diameter which is less than the first diameter to allow for a greater degree of deflection of deformation along proximal segment 54. In yet another alternative, member 50 may be fabricated from two or more different materials connected, attached, or otherwise formed together. In one example, distal segment 52 may be fabricated from a material having a high stiffness while proximal segment 54 may be fabricated from a material having a lower stiffness. Any of the above-described variations of member 50 and additional variations known to one of ordinary skill are intended to be within the scope of this disclosure.

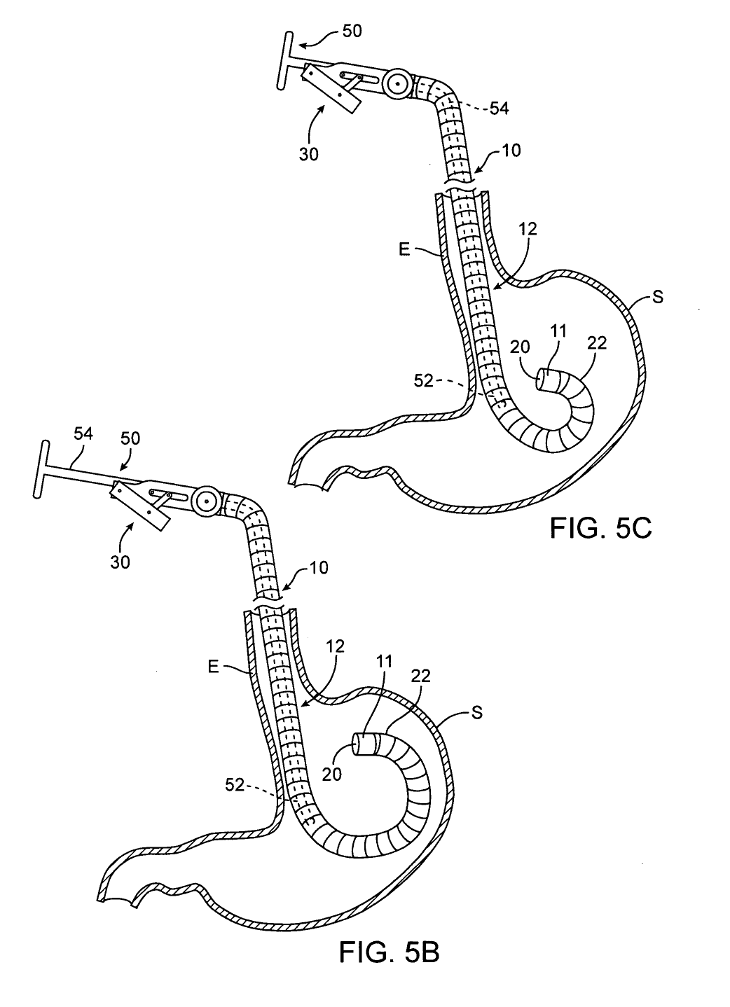

With reference to FIGS. 5, a method of varying the pivot of guide 12 with member 50 is described. As seen in FIG. 5A, guide 12 may be advanced per-orally and endoluminally into a patient's stomach S and retroflexed Ret such that distal outlet 20 of lumen(s) 13, or a distal region of the guide 12, is directed toward the body of the guide at a desired angle, for example, perpendicular to the body. Member 50 then may be advanced within a lumen 13 of guide 12.

As seen in FIG. 5B, distal segment 52 of member 50 is disposed at a desired distal position within a guide lumen 13, while proximal segment 54 conforms to the patient's anatomy and to the profile of guide 12, thereby facilitating advancement of the distal segment 52. As guide 12 is articulated or retroflexed, member 50 applies a restoring force to the guide that resists retroflexion or articulation of the guide proximal of the distal region of distal segment 52. Thus, member 50 alters the steering pivot of guide 12, such that the guide pivots about the distal end or distal region of segment 52. This may, for example, alter a degree of retroflexion Ret of the guide, as in FIG. 5B. However, the angle between distal outlet 20 of lumen(s) 13 (or a distal region of guide 12) and the body of guide 12 preferably remains unchanged as the pivot of the guide is varied.

As seen in FIG. 5C, the depth of insertion of distal segment 52 of member 50 within a lumen 13 of guide 12 may be dynamically altered to dynamically vary the steering pivot of guide 12. In FIG. 5C, member 50 is advanced deeper within guide 12 than in FIG. 5B, which shortens the steerable segment of guide 12 disposed distal of distal segment 52, thereby tightening the guide's radius of curvature in the retroflexed configuration. Likewise, retracting member 50 relative to guide 12 lengthens the steerable segment of the guide and increases the guide's radius of curvature when retroflexed. In this manner, the steering pivot of guide 12 is dynamically altered, and a length of the guide's steerable segment is dynamically varied from its natural length shown in FIG. 5A to any desired length shorter than that of FIG. 5A. As the steering pivot of guide 12 is dynamically altered or varied, the guide may be translated as desired, for example, to vary a length of the guide disposed within the body lumen, to alter a position of the distal region of the guide relative to the body lumen, etc. Although member 50 is described utilized with guide 12, which is comprised of multiple segments 14, member 50 and its variations may also be utilized with a number of conventional endoscopes having at least one working lumen therethrough. In such a variation, member 50 may be advanced and/or retracted through the endoscope lumen to alter the retroflexion of a steerable portion of the endoscope, if present.

Although member 50 illustratively has been advanced within a lumen 13 of guide 12 after the guide has been retroflexed, it should be understood that the member alternatively may be advanced within the lumen to vary the pivot of the guide while the guide is in a substantially straight or pliant configuration. Additionally or alternatively, member 50 may be advanced within a lumen 13 of guide 12 prior to advancement of the guide within a patient's body; thereafter, the guide and member may be advanced concurrently within the patient's body.

With member 50 disposed within a lumen 13, additional instruments or tools may be advanced through the lumen adjacent to member 50. Alternatively, guide 12 may comprise multiple lumens 13. Member 50 may be advanced within a first lumen 13, while additional instruments or tools may be advanced through one or more additional lumens 13.

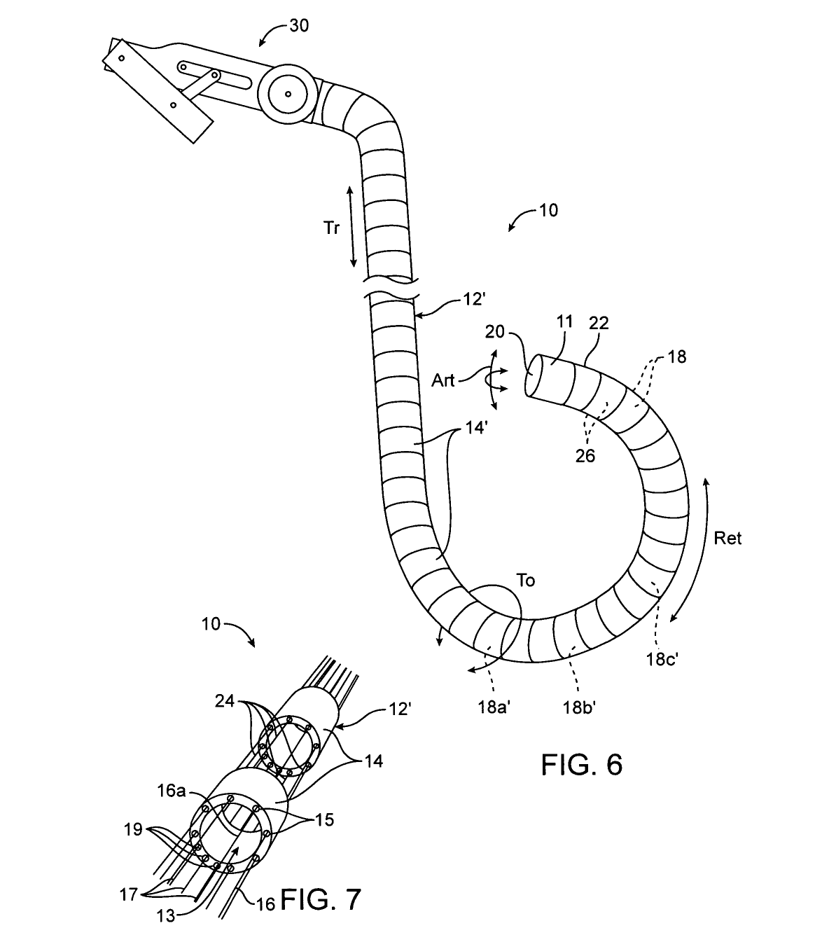

Referring now to FIGS. 6 and 7, an alternative variation of apparatus 10 is described. As seen in FIGS. 6 and 7, guide 12′ comprises a plurality of tensioning pivot wires 17 that extend through one or more pivot wire lumens 19 formed in the walls of segments 14′ of guide 12′. Wires 17 terminate at terminations 18′ within segments 14′ of guide 12′. Terminations 18′ of pivot wires 17 are disposed proximal of terminations 18 of tensioning wires 16. Terminations 18′ may, for example, be disposed at different positions along the length of guide 12′ to provide the medical practitioner with greater ability to dynamically vary the pivot of guide 12′.

The steering pivot of the guide may, for example, be altered by applying tension to one or more of the pivot wires 17, while concurrently applying tension to one or more steering wires 16 that terminate more distally than the pivot wires, and that are radially offset from the pivot wire about the circumference of guide 12′. When the steering wire(s) are about 180° offset from the pivot wire(s), as is steering wire 16a in FIG. 6, it is expected that the guide will pivot or retroflex about the distal-most termination 18′ of a tensioned pivot wire 17. Thus, variable pivoting may be achieved by controlling which wire(s) 17 are tensioned, e.g., by selecting the position of the distal-most termination 18′ of a wire 17 to which tension has been applied. In addition to varying the dynamics of retroflexion, other complex steering configurations may be achieved by tensioning various combinations of wires 16, 17 and/or 24. Although guide 12′ illustratively comprises a plurality of pivot wires 17 and a plurality of attendant terminations 18′, it should be understood that the guide alternatively may comprise any other number of pivot wires and terminations as practicable (for example, a single pivot wire and a single termination, two pivot wires and terminations, or more than three pivot wires and terminations), to provide the guide with any number of desired pivot positions or configurations.

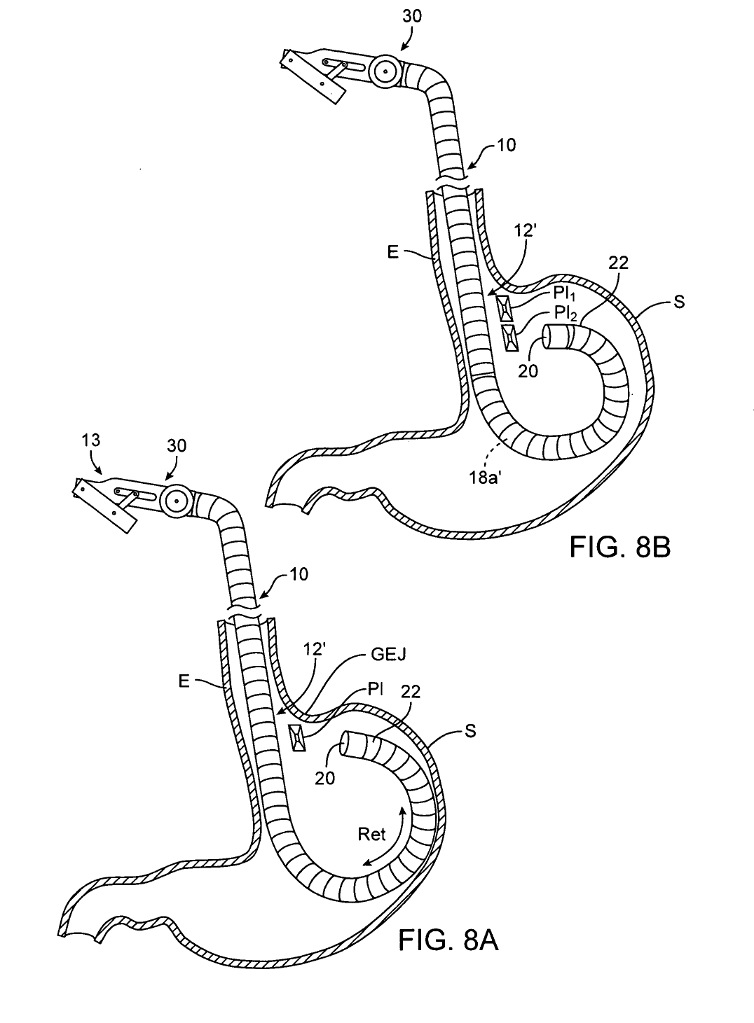

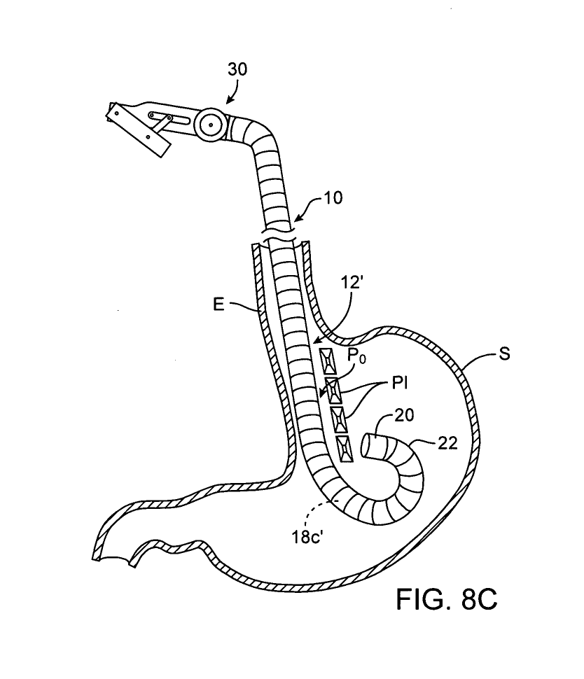

Referring now to FIGS. 8, an exemplary method of using the apparatus of FIGS. 6 and 7 to form a gastric pouch or partition is described. Guide 12′ may be advanced per-orally and endoluminally within a patient's stomach. The guide then may be retroflexed Ret as in FIG. 8A, such that outlet 20 of lumen 13 and/or a distal region of guide 12′ faces the body of guide 12′ just below gastroesophageal junction GEJ at a desired angle. Retroflexion may, for example, be achieved by applying tension to wire 16a (see FIG. 7). A first set of anterior and posterior tissue plications Pl₁ then may be formed, approximated and secured to one another within stomach S in the vicinity of outlet 20 of the lumen of guide 12′, for example, by articulating Art tip 22 of the guide via one or more steering wires 24 and/or by manipulating gastric tissue via instruments or tools advanced through one or more lumens 13 of the guide.

As seen in FIG. 8B tension may be applied to one or more pivot wires 17, e.g., via handle 30, to alter the pivot of the guide, while preferably substantially maintaining the desired angle at which outlet 20 of lumen 13 and/or the distal region of guide 12′ faces the body of the guide. For example, tension may be applied to a wire 17 having proximal-most termination 18a′, thereby altering the pivot a relatively small amount while shortening the retroflexed length of guide 12′ disposed distal of the termination and tightening the guide's retroflexed radius of curvature. Concurrently, guide 12′ may be translated or retracted. Such coordinated motion positions outlet 20 and/or a distal region of guide 12′ facing the body of the guide just below the first approximated plications Pl₁ of FIG. 8A. Additional anterior and posterior plications Pl₂ may be formed, approximated and secured in the vicinity of this new position of outlet 20.

As seen in FIG. 8C, this procedure may be repeated as desired. For example, by applying tension to pivot wires 17 having progressively more distal terminations 18′, e.g. terminations 18b′ and/or 18c′, and concurrently translating guide 12′ within stomach S as needed, a degree of pivot variation of the guide may be progressively increased such that the retroflexed length of guide 12′ is progressively shortened, the guide's retroflexed radius of curvature is progressively tightened, and outlet 20 or a distal region of the guide is positioned at progressively lower positions within stomach S. Plications Pl may be formed, approximated and secured at these progressively lower positions within the stomach to form gastric partition or pouch Po. In this manner, regions of the tissue which may be more difficult to access because of limited space within the stomach, e.g., the regions of tissue relatively near the lesser curvature, may be effectively accessed as described by guide 12′ for performing a treatment. As will be apparent, variable pivoting of guide 12′ may be used to form pouch Po in a different manner or in a different sequence of steps. Furthermore, variable pivoting may be utilized for alternative gastrointestinal indications.

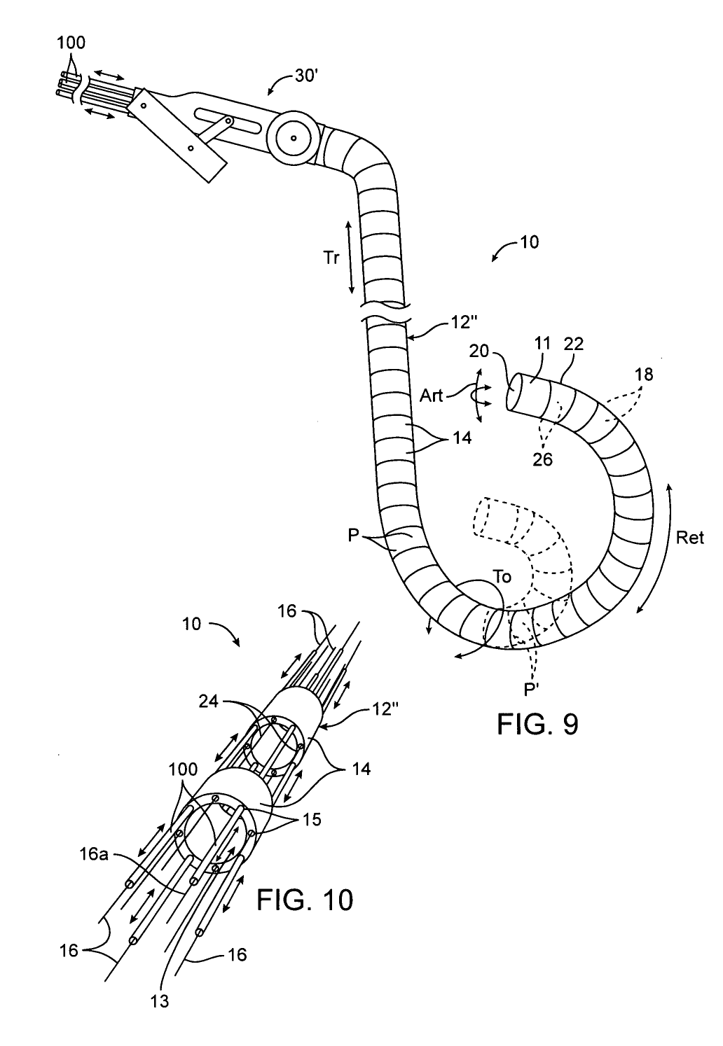

Referring now to FIGS. 9 and 10, another variation of the steerable guide of FIG. 1 is described. Guide 12″ is similar to guide 12 of FIGS. 1 and 2, except that stiffening elements 100 are disposed coaxially or alongside one or more tensioning wires 16 and/or steering wires 24 that pass through wire lumens 15 and terminate within guide 12″ at terminations 18 or 26. Elements 100 are configured for translation within lumens 15, such that a position of the distal end of the elements along a longitudinal axis of guide 12″ may be altered to alter a steering pivot of the guide, as described hereinafter. In one variation, elements 100 may be fabricated from hypotubes, for example, Nitinol or polymeric hypotubes. In another variation, elements 100 may comprise flexible coils.

As seen in FIG. 9, element(s) 100 slidably extend through handle 30′ into lumen(s) 15 of guide 12″. The distal region(s) of element(s) are set at an initial position P within lumen(s) 15, and wires 16 or 24 extend distally of position P to terminations 18 or 26, respectively. In this initial configuration, steering of guide 12″ may be achieved by selectively applying radially unbalanced forces to segments 14 via one or more of the wires, e.g., via wire 16a. Element(s) 100 resist steering moments applied by the wire to the guide, such that position P acts as the steering pivot of the guide.

As illustrated in dotted profile in FIG. 9, element(s) 100 may be translated within lumen(s) 15 to alter the steering pivot of guide 12′. In FIG. 9, the element(s) illustratively are advanced relative to the guide to position the distal region(s) of the element(s) at position P′. Thereafter, position P′ serves as the steering pivot of the guide. This shortens the steerable length of the guide and the steering dynamics of the guide's tip. As will be apparent, retraction of elements 100 likewise extends the steerable length of the guide. In this manner, the steering pivot of guide 12″ may be altered as desired.

Although preferred illustrative embodiments of the present invention are described hereinabove, it will be apparent to those skilled in the art that various changes and modifications may be made thereto without departing from the invention. It is intended in the appended claims to cover all such changes and modifications that fall within the true spirit and scope of the invention.

What is claimed is:

1. Apparatus for obtaining endoluminal access, the apparatus comprising:
a steerable endoscopic guide; and
a pivot element adapted to alter a position of a pivot along the guide.

2. The apparatus of claim 1, wherein the steerable endoscopic guide comprises a plurality of discrete segments.

3. The apparatus of claim 2, further comprising a plurality of tensioning wires coupled to the guide for steering or rigidizing the guide by torquing or compressing the discrete segments.

4. The apparatus of claim 2, wherein the guide further comprises at least one lumen extending through the discrete segments.

5. The apparatus of claim 1, wherein the pivot element comprises an elongate stiffening member slidably positioned relative to the guide, wherein a terminal end or distal portion of the stiffening member defines the pivot of the guide.

6. The apparatus of claim 3, wherein the pivot element comprises at least one pivot wire coupled to the guide proximal of at least one of the tensioning wires for varying the pivot of the guide.

7. The apparatus of claim 3 further comprising at least one steering wire coupled to the guide distal of at least one of the tensioning wires to facilitate articulation of the guide.

8. The apparatus of claim 3, wherein the tensioning wires facilitate retroflexion of the guide to a configuration wherein an outlet of the lumen extending through the guide faces a body portion of the guide.

9. The apparatus of claim 8, wherein retroflexion of the guide disposes the outlet of the lumen perpendicularly to the body portion of the guide.

10. The apparatus of claim 8, wherein the pivot element is configured to alter a radius of curvature of the retroflexed guide.

11. The apparatus of claim 8, wherein the guide is configured to maintain an angle between the outlet of the lumen and the body portion of the guide upon altering the position of the pivot along the guide.

12. The apparatus of claim 1 further comprising instruments or tools configured for passage through at least one lumen defined through the guide.

13. The apparatus of claim 12, wherein the instruments or tools are configured to manipulate tissue.

14. The apparatus of claim 12, wherein the instruments or tools are configured to approximate plicated tissue.

15. The apparatus of claim 12, wherein the instruments or tools are configured to secure plicated tissue.

16. The apparatus of claim 5, wherein the stiffening member comprises a relatively rigid distal portion and a relatively flexible proximal portion.

17. The apparatus of claim 16, wherein the relatively flexible proximal portion is configured to conform to a shape of the guide or of a patient's anatomy.

18. The apparatus of claim 3, wherein the pivot element comprises at least one stiffening element disposed coaxially or alongside at least one of the plurality of tensioning wires for varying the pivot of the guide.

19. The apparatus of claim 18, wherein the at least one stiffening element comprises a hypotube.

20. The apparatus of claim 18, wherein the at least one stiffening element comprises a flexible coil.

21. A method for obtaining endoluminal access, the method comprising:
endoluminally positioning a steerable guide within a patient;
steering the guide into a desired configuration; and
altering a position of a pivot element along the guide to change a steering configuration of the guide.

22. The method of claim 21 further comprising performing a first diagnostic or therapeutic medical procedure with instruments delivered via the guide after endoluminally positioning a steerable guide.

23. The method of claim 22 further comprising performing a second diagnostic or therapeutic medical procedure with the instruments while the guide is disposed in an alternate steering configuration.

24. The method of claim 21, wherein endoluminally positioning a steerable guide within a patient further comprises endoluminally advancing the guide into the patient's stomach.

25. The method of claim 24, further comprising partitioning the patient's stomach with instruments delivered via the guide.

26. The method of claim 21, wherein steering the guide into a desired configuration comprises retroflexing the guide such that a distal outlet of the guide faces a body portion of the guide.

27. The method of claim 26, wherein altering a position of a pivot element along the guide further comprises altering a degree of retroflexion of the guide.

28. The method of claim 21, wherein altering a position of a pivot element along the guide further comprises translating a stiffening member within a lumen of the guide.

29. The method of claim 21, wherein altering a position of a pivot element along the guide further comprises applying tension to a pivot wire that is coupled to the guide distal of the guide's pivot element.

30. The method of claim 21, wherein steering the guide further comprises applying tension to a wire or cable attached to the guide, and wherein altering a position of a pivot element along the guide further comprises translating a stiffening element along the wire or cable.

31. Apparatus for obtaining endoluminal access, the apparatus comprising:
a steerable endoluminal guide comprising a plurality of discrete segments;
plurality of tensioning wires coupled to the guide for steering or shape-locking the guide by torquing or compressing the discrete elements, the guide being steerable such that an outlet of a lumen passing through the guide faces a body portion of the guide; and
a pivot element configured to alter a steering configuration of the guide by altering a position of the pivot along the guide.
